(19) United States
(12) Patent Application Publication
(10) Pub. No.: US 2026/0083961 A1
(43) Pub. Date: Mar. 26, 2026

(54) MEDICAL IMPLANT IN THE FORM OF A WRAP SLEEVE

(71) Applicant: Neuroloop GmbH, Freiburg (DE)

(72) Inventors: Fabian Kimmig, Freiburg (DE); Tim Boretius, Freiburg (DE); Max Eickenscheidt, Freiburg (DE)

(21) Appl. No.: 19/109,210

(22) PCT Filed: Sep. 4, 2023
(86) PCT No.: PCT/EP2023/074158

§ 371 Date: Mar. 6, 2025

(30) Foreign Application Priority Data
Sep. 7, 2022 (DE) 10 2022 122 699.9

(51) Int. Cl. A61N 1/05

(52) U.S. Cl. CPC A61N 1/0556

(57) ABSTRACT

A medical implant forms a sleeve for arranging about a nerve fiber bundle. The sleeve has a film-like substrate with a first section that converts into a hollow-cylindrical shape. A first end of the first section rests against a first surface of the first section in the wound state. The first surface faces an area enclosed by the wound first section. A second end lies opposite the first end and in the wound state is arranged laterally to a second surface of the first section. The second surface faces away from the first surface and from the radially enclosed area. An area of the first section end is monolithically connected with a second section, which in the wound state is adjacent to the second surface of the first section. The first and second sections have the set shape, with a winding direction oriented oppositely relative to the first end.

### CROSS-REFERENCE TO RELATED APPLICATIONS

This application is the United States national stage entry of International Application No. PCT/EP2023/074158, filed on Sep. 4, 2023, and claims priority to German Application No. 10 2022 122 699.9, filed on Sep. 7, 2022. The contents of International Application No.

PCT/EP2023/074158 and German Application No. 10 2022 122 699.9 are incorporated by reference herein in their entireties.

### FIELD

The invention relates to a medical implant in the form of a wrap sleeve for arranging about a nerve fiber bundle, comprising a flat biocompatible film-like substrate with at least one first flat substrate section that can be converted into a hollow-cylindrical shape by means of a winding direction which is specified by a set shape that is inherent to the material about a spatial axis and has a first flat substrate section end which rests against a first surface of the first flat substrate section in the wound state, said surface being oriented so as to face an area radially enclosed by the wound first flat substrate section, and a second flat substrate section end which lies opposite the first flat substrate section end and in the wound state is arranged laterally to a second surface of the first flat substrate section, said second surface facing away from the first surface and orientated facing away from the area radially enclosed by the wound first flat substrate section.

### BACKGROUND

So-called wrap sleeves, also known as cuff electrodes, are used for the purpose of intracorporeal application of electrical stimulation signals along a nerve fiber bundle. A particularly preferred cuff electrode is disclosed in document EP 3 204 105 B1. For locally wrapping around a nerve fiber bundle, cuff electrodes have a flat biocompatible film-like substrate, onto the surface of which an electrode arrangement is applied for the application of electrical signals, that is brought into direct surface contact with the epineurium of a nerve fiber bundle. For this, the flat film-like substrate exhibits an intrinsically ingrained mechanical tension such that in an otherwise force-free state, the otherwise preferably rectangular flat film-like substrate can wind or roll up autonomously about a winding axis, thereby forming a straight hollow cylindrical shape. Usually, the flat film-like substrate has a flat, rectangular shape at least in the area of the material-inherent pre-tensioning, with a free flat substrate lateral edge which is guided at least once, preferably several times, about a winding axis, wherein the film-like carrier substrate multiply forms hollow cylindrically shaped flat substrate windings that loosely rest against each other. The electrode arrangement is applied in the area of the flat substrate surface directly facing the volume enclosed by the wrap sleeve in order to thus, in the implanted state and locally applied about a nerve fiber bundle, directly contact the surface of the epineurium of the nerve fiber bundle. Multiple wrapping of the flat substrate about the nerve fiber bundle supports and/or improves the area contact or pressing pressure between the electrode arrangement and the nerve fiber bundle, through which the mechanical hold on the outer circumference of the nerve fiber bundle is improved with an increasing number of windings.

The radially outermost winding of the flat substrate transitions in one piece into a mechanically non-pretensioned, i.e. otherwise planar flat substrate section, onto which an electrical contact arrangement is usually applied, via which electrical input and output lines connected to the electrode arrangement can be connected via a corresponding conductor structure to an implantable electrical supply unit in which both means for electrical energy supply and also for controlling and operating the cuff electrode are accommodated.

The hitherto usual practice of implanting a cuff electrode requires great skill and steady hands on the part of the operating surgeon, particularly as in the normal, i.e., force-free, state the cuff electrode autonomously assumes the aforementioned hollow cylindrical wound state. To convert the cuff electrode into a unrolled or open state for applying to the outer circumference of a nerve fiber bundle, on the one hand the cuff electrode needs to be picked up at its mechanically non-pre-tensioned flat area, preferably using tweezers, and on the other hand, the cuff-like wound flat substrate area needs to be stretched flat under the effect of a force opposite to the material-inherent winding direction, so that the film-like flat substrate takes on an unrolled flat shape.

Suitable for this is wire-like instrument bent into an “L” shape, the shorter L arm of which is passed on one side completely through the rolled-up cuff electrode along the winding axis. Subsequently, through dosed tensile force along the longer L arm of the instrument and with corresponding counter-holding by means of the tweezers, the wrap sleeve is converted into an elongated, i.e. stretched shape.

The operating surgeon must perform the procedure explained above with two hands, and the utmost concentration is required both for placing the cuff electrode along the nerve fiber bundle as well as for the process of unwinding the cuff electrode, particularly as even slightly too wide spreading of the windable flat substrate results in the instrument sliding off or detaching from the stretched flat substrate, through which the flat substate spontaneously assumes the rolled up or wound up state again. Especially under real operating conditions, in which only limited spatial, visual and time conditions prevail, a high degree of manual dexterity and surgical experience is demanded of the operating surgeon. Furthermore, failed attempts or unsuccessful applications of the cuff electrode along a nerve fiber bundle constitute avoidable stresses for the patient.

From document DE 10 2018 207 709 A1 a device for the extra-neuronal fastening of a medical implant with a biocompatible flat substrate can be derived, which has a first substrate section designed as a wrap sleeve that has a free section end that through winding the first substrate section about a spatial axis is loosely covered radially by the wound first substrate section at least in one layer, and also envisages a second substrate section not wound around the spatial axis and adjoining the first substrate section in one piece, that can be directly or indirectly connected to a connection structure leading away from the medical implant. In the area of the section end, at least one means is arranged that in the wound state of the first substrate section about the spatial axis enters into a joining connection with the first substrate section or with the second substrate section.

### SUMMARY

The object of the invention is to further develop a medical implant in the form of a wrap sleeve for arranging about a nerve fiber bundle, comprising a flat biocompatible film-like substrate with at least one first flat substrate section that can be converted into a hollow-cylindrical shape by means of a winding direction which is specified by a set shape that is inherent to the material about a spatial axis and has a first flat substrate section end which rests against a first surface of the first flat substrate section in the wound state, said surface being oriented so as to face an area radially enclosed by the wound first flat substrate section, and a second flat substrate section end which lies opposite the first flat substrate section end and in the wound state is arranged laterally to a second surface of the first flat substrate section, said second surface facing away from the first surface and orientated facing away from the area radially enclosed by the wound first flat substrate section, in such way that manipulation by an operating surgeon when implanting and applying the wrap sleeve about a nerve fiber bundle is made significantly easier and incorrect manipulations and incorrect placements are largely ruled out. In addition, it should be possible to safety and expertly manipulate the wrap sleeve in surgically confined spatial conditions.

According to the solution, a medical implant in the form of a wrap sleeve for arranging around a nerve fiber bundle is characterized in that the first and second flat substrate sections each have the set shape that is inherent to the material with a respective winding direction that is oriented oppositely relative to the first flat substrate section.

Preferably the first flat substrate section end is formed through folding the flat biocompatible film-like substrate in the manner of a folded edge, so that the first and second flat substrate sections rest flat directly against each other. Not necessarily, but preferably, both flat substrate sections are identical in shape and size, or identically dimensioned, so that in the wound state they rest flat and in full against each other.

Also conceivable is an embodiment of the second flat substrate section that is smaller in area, e.g. strip-shaped, than the first flat substrate section, that at least locally projects beyond the second flat substrate section end in order to provide unhindered access to the second flat substrate section, for example by means of a gripping instrument, for example tweezers.

The first and the second flat substrate section each have a set shape that is inherent to the material so that both flat substrate sections that are connected over the common folded edge autonomously roll up helically about a common winding axis, wherein the common folded edge that is equivalent to the first flat substrate section end, is arranged internally with regard to the forming hollow cylindrical winding shape. If both flat substrate sections are pulled apart at their flat substrate section ends opposite the folded edge, for example by way of two tweezers, it becomes clear that both flat substrate section ends that are connected via the first flat substrate section end or the folded edge and in the stretched state are largely arranged opposite it, have a set shape inherent that is inherent to the material, have an opposite winding direction.

In order to guarantee safe and reliable handling of the medical implant for the purpose of implantation or application along a nerve fiber bundle and, in addition, to facilitate this, the second flat substrate section end of the first flat substrate section is monolithically connected to a fourth flat substrate section which at least in sections projects beyond the first flat substrate section in the form of a tab, tongue or lug.

The second flat substrate section comprise a third flat substrate section end which is opposite the first flat substrate section end designed as a folded edge and which in the wound state at least partially projects beyond the second flat substrate section end and/or along which a fifth flat substrate section is monolithically connected, which at least in sections projects beyond the second flat substrate section in the form of a tab, tongue or lug.

In each case, the medical implant can be gripped with a gripping instrument, for example tweezers, or manually on the fourth and fifth tab or tongue-like flat substrate section, and through diametral pulling apart can be converted from the wound state into an open state in which the medical implant can be comfortably locally applied around a nerve fiber bundle.

### BRIEF DESCRIPTION OF THE DRAWINGS

The invention is described as an example below without limitation of the general inventive idea by way of examples of embodiment with reference to the drawings. In these:

FIG. 1a, b shows a comparison of a cuff electrode arrangement according to the prior art, see a), with a cuff electrode arrangement according to the solution, see b), and

FIGS. 2a-2g show sequence images for producing the cuff electrode according to the invention as well as application of the cuff electrode along a nerve fiber bundle.

### DETAILED DESCRIPTION

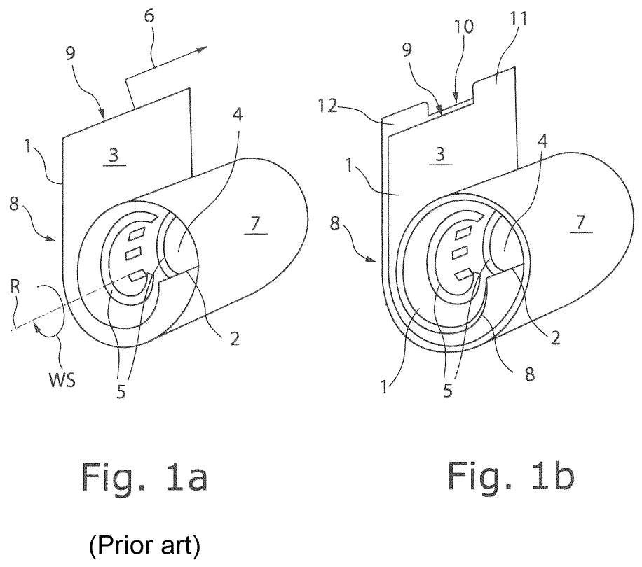

FIG. 1a shows a cuff electrode that is known per se, with a flat substrate section 1 made of a flat biocompatible film-like substrate, which through a shape that is inherent to the material autonomously assumes a hollow cylindrical winding shape with a predetermined winding direction WS about a spatial axis R. In this wound state, the first flat substrate section 1 with its surface 3 facing the drawing plane radially encloses a cylindrically shaped area 4 from outside, through which, in the implanted state of the cuff electrode, a nerve fiber bundle projects in manner so as to fill the area. On the surface 3 of the first flat substrate section 1, in the area of the first winding radially enclosing the area 4, an electrode arrangement 5 is applied that can come into direct contact with the epineurium of a nerve fiber bundle. The first flat substrate section 1 is preferably of a flat rectangular shape and has a first flat substrate section end 2, which, as shown in FIG. 1a, in the wound state loosely rests against the surface 3 of the flat substrate 1. In the wound state, the cuff electrode has at least one complete winding, preferably multiple flat substrate windings form in order to thus obtain a winding shape of the cuff electrode that is stable in the long term.

For the sake of completeness, it is pointed out that the electrode arrangement 5 is contacted via electrical input and output leads extending within the flat substrate that are not shown in further detail, connected via a further connection structure 6 connected to the first surface substrate section 1 with an implantable supply unit that is not shown further and which is not the subject of the application.

In contrast, shown in FIG. 1b is a preferred form of embodiment of the medical implant, designed according to the solution in the form of a wrap sleeve or cuff electrode, which in addition to the known cuff electrode illustrated in FIG. 1a, is, along its first flat substrate section end 2, monolithically connected with a further, the so-called second flat substrate section 7, which in the wound state loosely rests against the surface 8 facing away from the surface 3 visible in FIG. 1b.

In the shown case, in FIG. 1b the second flat substrate section 7 is of the same shape and same dimensions as the first flat substrate section 1 and with this jointly radially encloses the inner area 4, wherein the electrode arrangement 5 applied on the first flat substrate section remains facing the inner area 4 in an unchanged manner.

The first and the second flat substrate section 1, 7 each have a second or third flat substrate section end 9, 10 opposite the first flat substrate section end 2, which are each monolithically connected to a tongue or lug-shaped flat substrate projection 11, 12. The flat substrate projections 11, 12 are intended for simpler and more secure handling of the cuff electrode, preferably using a gripping instrument, for example with the aid of tweezers.

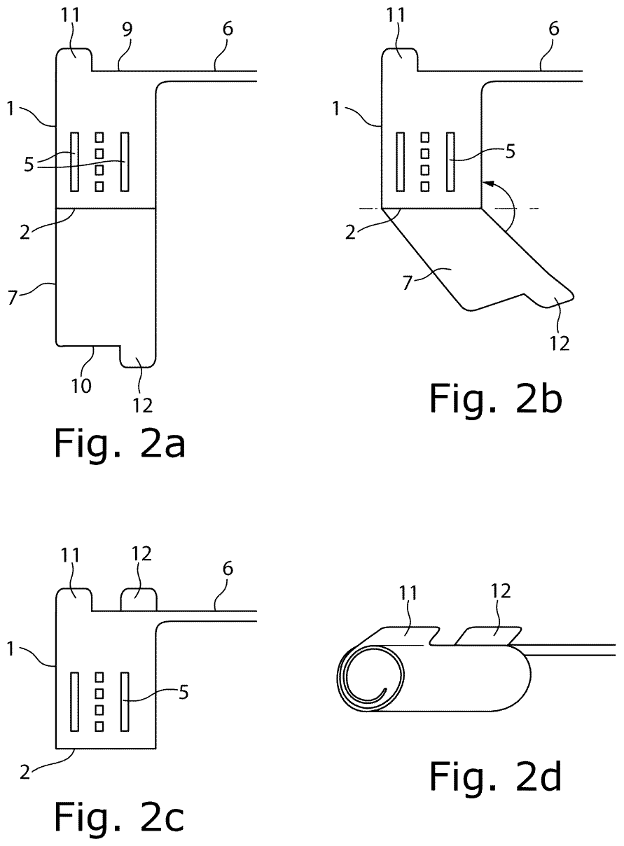

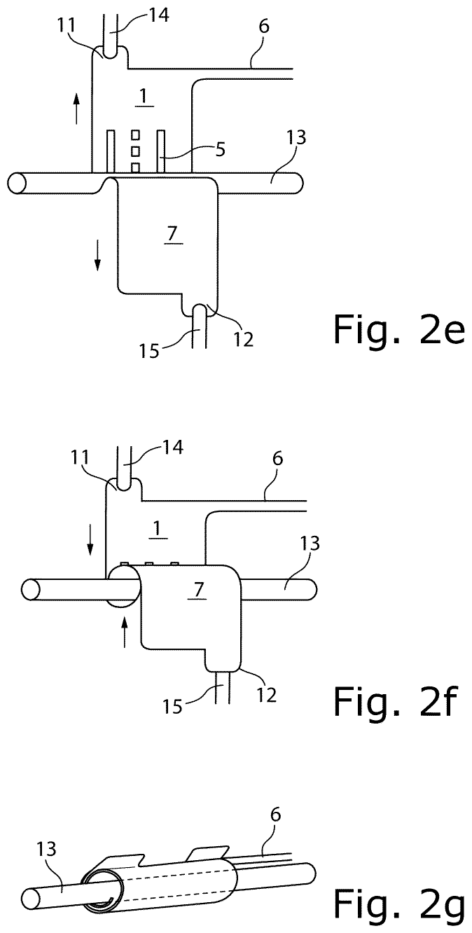

FIG. 2a to g illustrate the production and handling of the cuff electrode for the purpose of application around a nerve fiber bundle.

FIG. 2a shows a flat biocompatible film-like substrate that is flat and rectangular in design. The upper half of the flat substrate in the illustration according to FIG. 2, represents the first flat substrate section 1 onto the surface 3 of which the electrode arrangement 10 is applied. The lower half connected with the first flat substrate section 1 and corresponding to the second flat substrate section 7 is, as in the presented case, of the same shape and same dimensions as the first flat substrate section 1. Both flat substrate sections 1, 7 envisage at their respective flat substrate section ends 9, 10 the previously explained tab or tongue-shaped flat substrate section projections 11, 12. The second flat substrate section 7 can also be designed to be smaller in area, essential being that in the wound state the second flat substrate section 7 locally projects beyond the first flat substrate section 1 at its flat substrate section end 9.

For the purpose of electrical energy and signal transmission and supplying the electrode arrangement 10, on the flat substrate section 1 a connection structure is also applied that is integrated in the flat substrate and leads to an implantable supply unit which is not shown further.

The first and second flat substrate section 1, 7 are monolithically connected to each other by way of the so-called first flat substrate section end 2, which as shown further, is in the form of a folded edge.

Thus, in FIG. 2b, the second flat substrate section 7 is folded backwards along and around the first flat substrate section end 2, see the arrows in FIG. 2b, so that on the rear of the electrode arrangement 10, the second flat substrate section 7 comes into flat contact on the first flat substrate section 1, see FIG. 2c. The two first and second flat substrate sections 1, 7 flatly resting against each other are then, starting from their common flat substrate section end designed as a folded edge, wound around a spatial axis in such a way that the electrode arrangement 10 remains oriented toward the area radially enclosed during the winding. The wound state is shown in FIG. 2d. In this illustration, the entire surface area of the first and second flat substrate section 1, 7 is rolled up in the form of a two-layer cylinder. In order to ingrain the cylindrical shape into the flat biocompatible film-like substrate in a manner that is inherent to the material, the wound cuff electrode in the form illustrated in FIG. 2d undergoes a tempering process.

For the purpose of handling and applying the cuff electrode along a nerve fiber bundle 13, see FIG. 2e, the cuff electrode is converted into a stretched or elongated state in that an operating surgeon grips the flat substrate projections 11, 12 with the aid of a suitable gripping instrument 14, 15, for example tweezers, and converts the cuff electrode into a stretched state as shown in FIG. 2e. In this stretched or open state, the operating surgeon is able to apply the cuff electrode at a defined surface area of a nerve fiber bundle 13. Through reducing the tensile forces acting on the cuff electrode, the cuff electrode winds around the nerve fiber bundle 13 autonomously, see FIG. 2f. The autonomous rolling up of the cuff electrode around the nerve fiber bundle 13 can be controlled in a dosed manner in that the operating surgeon reduces the tensile or retaining forces in a controlled way using the tweezers 14, 15.

As soon as the cuff electrode has been placed at the right point along the nerve fiber bundle 13, the cuff electrode remains stable in the long term due to the material-inherent restoring force predominating in the double-layer cuff electrode. This state is shown in FIG. 2g.

### LIST OF REFERENCE NUMBERS

1 Flat substrate
2 First flat substrate end
3 Surface
4 Area
5 Electrode arrangement
6 Connection structure
7 Second flat substrate end
8 Opposite surface
9 Second flat substrate section end
10 Third flat substrate section end
11 Flat substrate projection
12 Flat substrate projection
13 Nerve fiber bundle
14, 15 Gripping instrument, for example tweezers

1. -7. (cancelled).

8. A medical implant comprising a wrap sleeve for arranging about a nerve fiber bundle, the wrap sleeve comprising:
a flat biocompatible film-like substrate made of a material and having at least one first flat substrate section that is convertible into a hollow-cylindrical shape by a winding direction which is specified by a set shape that is inherent to the material about a spatial axis and has a first flat substrate section end which rests against a first surface of the at least one first flat substrate section in a wound state, the first surface being oriented so as to face an area radially enclosed by the at least one first flat substrate section, and a second flat substrate section end which lies opposite the first flat substrate section end and in the wound state is arranged laterally to a second surface of the at least one first flat substrate section, said second surface facing away from the first surface and orientated facing away from the area radially enclosed by the at least one first flat substrate section,
at least one area of the first flat substrate section end being monolithically connected with a second flat substrate section, which in the wound state is adjacent to the second surface of the at least one first flat substrate section,
the at least one first flat substrate section and the second flat substrate section each having the set shape that is inherent to the material, with the winding direction oriented oppositely relative to the first flat substrate section end.

9. The medical implant according to claim 8, wherein through folding of the flat biocompatible film-like substrate, the first flat substrate section end is a folded edge.

10. The medical implant according to claim 8, wherein the second flat substrate section has a third flat substrate section end which at least partially projects beyond the at least one first flat substrate section in the wound state along the second flat substrate section end.

11. The medical implant according to claim 10, wherein:
the second flat substrate section end of the at least one first flat substrate section is monolithically connected along at least one section to a flat substrate section projection, which at least partially projects beyond the at least one first flat substrate section, and
the third flat substrate section end of the second flat substrate section is monolithically connected along at least one section to a further flat substrate section projection, which at least partially projects beyond the at least one first flat substrate section.

12. The medical implant according to claim 11, wherein the flat substrate section projection and the further flat substrate section projection comprise tabs.

13. The medical implant according to claim 8, wherein the wrap sleeve is designed as a cuff electrode sleeve with an electrode arrangement that is arranged on and within the at least one first flat substrate section and on the first surface has at least one freely accessible electrode contact surface.

14. The medical implant according to claim 8, wherein the flat biocompatible film-like substrate as well as the set shape that is inherent to the material in the at least one first flat substrate section and the second flat substrate section are selected so that, through manually performed application, the medical implant is, through ending an application of tensile force, convertible autonomously from an elongated state, in which the at least one first flat substrate section and the second flat substrate section are stretched along a plane in opposite directions under tensile force, into the wound state.
